(19) United States
(12) Patent Application Publication
(10) Pub. No.: US 2025/0345314 A1
(43) Pub. Date: Nov. 13, 2025

(54) STEM CELL CONJUGATED WITH RAPAMYCIN-CONTAINING DRUG CARRIER AND USES THEREOF

(71) Applicants: PUSAN NATIONAL UNIVERSITY INDUSTRY-UNIVERSITY COOPERATION FOUNDATION, Busan (KR); RESEARCH & BUSINESS FOUNDATION SUNGKYUNKWAN UNIVERSITY, Suwon-si (KR); KANGSTEM BIOTECH CO., LTD., Seoul (KR)

(72) Inventors: Hyung-Sik KIM, Yangsan-si (KR); Jee-Heon JEONG, Suwon-si (KR); Junhyeung PARK, Suwon-si (KR); Soyeong PARK, Yangsan-si (KR); Min-Jung KANG, Yangsan-si (KR)

(21) Appl. No.: 19/056,005

(22) Filed: Feb. 18, 2025

(30) Foreign Application Priority Data
May 10, 2024 (KR) 10-2024-0061919

(51) Int. Cl. A61K 31/436; A61K 47/59; A61K 47/69

(52) U.S. Cl. CPC A61K 31/436; A61K 47/593; A61K 47/6901

(57) ABSTRACT

The present disclosure relates to a stem cell conjugated with a rapamycin-containing drug carrier, and more particularly, to uses of the stem cell for treating pulmonary fibrosis and inhibiting fibrosis. According to the present disclosure, it was experimentally confirmed that the stem cell conjugated with the rapamycin-containing drug carrier had an excellent effect of inhibiting the expression of fibrosis-related proteins in pulmonary fibroblasts induced with fibrosis. Therefore, the stem cell conjugated with the rapamycin-containing drug carrier of the present disclosure may be used in various fields of research related to fibrosis and treatment of pulmonary fibrosis.

### CROSS-REFERENCE TO RELATED APPLICATIONS

This application is based on and claims priority from Korean Patent Application No. 10-2024-0061919, filed on May. 10, 2024, with the Korean Intellectual Property Office, the disclosure of which is incorporated herein in its entirety by reference.

### TECHNICAL FIELD

The present disclosure relates to a stem cell conjugated with a rapamycin-containing drug carrier, and more particularly, to uses of the stem cells for treating pulmonary fibrosis and inhibiting fibrosis.

### BACKGROUND

Idiopathic pulmonary fibrosis is a chronic, irreversible interstitial lung disease characterized by progressive pulmonary fibrosis. The idiopathic pulmonary fibrosis market is showing a rapid growth trend of about 13.9% of average annual growth rate, and the market growth is expected to accelerate due to the aging population. The pulmonary fibrosis is an incurable disease with a very poor prognosis, with an average life expectancy of 3 to 5 years after diagnosis, leading to death from respiratory failure and heart disease, and there is no therapy once fibrosis has progressed.

Currently, representative therapeutic agents for pulmonary fibrosis include pirfenidone and nintedanib. The pirfenidone inhibits collagen synthesis and fibroblast proliferation that are increased by TGF-β stimulation, and the nintedanib delays the progression of fibrosis by blocking a tyrosine kinase receptor signaling pathway to prevent the production of fibrosis-related growth factors. However, these therapeutic agents only have the effect of slightly delaying the progression of fibrosis and cannot return fibrotic tissue to normal tissue. In addition, there is the problem of stomach-related side effects, making it difficult to continuously take the therapeutic agents. Therefore, there is a need for a treatment technology for pulmonary fibrosis based on a mechanism that can reverse fibrosis that has already occurred or dramatically inhibit fibrosis that is in progress.

### SUMMARY

Accordingly, the present inventors confirmed that rapamycin inhibited the expression of collagen, a fibrosis-related factor, without affecting the survival rate of mesenchymal stem cells while studying the treatment of pulmonary fibrosis. In addition, rapamycin increased the production of anti-fibrotic paracrine factors from mesenchymal stem cells. Based on the results, the present disclosure was completed by preparing ‘mesenchymal stem cells conjugated with a rapamycin-containing drug carrier coated with polydopamine on the surface’ and confirming its anti-fibrosis effect.

The present disclosure has been made in an effort to provide a stem cell-drug carrier including a stem cell conjugated with a rapamycin-containing drug carrier on the cell surface.

The present disclosure has also been made in an effort to provide a composition comprising the stem cell-drug carrier.

The present disclosure has also been made in an effort to provide a pharmaceutical composition for preventing or treating pulmonary fibrosis including the stem cell-drug carrier.

The present disclosure has also been made in an effort to provide a reagent composition for inhibiting fibrosis including the stem cell-drug carrier.

The present disclosure has also been made in an effort to provide a pharmaceutical composition for preventing or treating pulmonary fibrosis including a stem cell primed with rapamycin.

The present disclosure has also been made in an effort to provide a reagent composition for inhibiting fibrosis including a stem cell primed with rapamycin.

The present disclosure has also been made in an effort to provide a pharmaceutical composition for preventing or treating pulmonary fibrosis including rapamycin and a stem cell.

The present disclosure has also been made in an effort to provide a reagent composition for inhibiting fibrosis including rapamycin and a stem cell.

The present disclosure has also been made in an effort to provide a method for preparing a cell therapy product for preventing or treating pulmonary fibrosis including (a) preparing a rapamycin-containing drug carrier by mixing and homogenizing rapamycin and a polymer; and (b) preparing a stem cell conjugated with the rapamycin-containing drug carrier by culturing the rapamycin-containing drug carrier prepared in step (a) and the stem cell.

An exemplary embodiment of the present disclosure provides a stem cell-drug carrier including a stem cell conjugated with a rapamycin-containing drug carrier on the cell surface.

In addition, another exemplary embodiment of the present disclosure provides a composition comprising the stem cell-drug carrier.

In addition, another exemplary embodiment of the present disclosure a pharmaceutical composition for preventing or treating pulmonary fibrosis including the stem cell-drug carrier.

In addition, another exemplary embodiment of the present disclosure provides a reagent composition for inhibiting fibrosis including the stem cell-drug carrier.

In addition, another exemplary embodiment of the present disclosure provides a method for treatment or inhibiting of fibrosis, comprising administering the stem cell-drug carrier.

In addition, yet another exemplary embodiment of the present disclosure provides a method for treatment or inhibiting of fibrosis, comprising administering a stem cell primed with rapamycin to a subject in need thereof.

In addition, yet another exemplary embodiment of the present disclosure provides a method for treatment or inhibiting of fibrosis, comprising administering a rapamycin and a stem cell to a subject in need thereof

In addition, yet another exemplary embodiment of the present disclosure provides a method for preparing a cell therapy product for preventing or treating pulmonary fibrosis including (a) preparing a rapamycin-containing drug carrier by mixing and homogenizing rapamycin and a polymer; and (b) preparing a stem cell conjugated with the rapamycin-containing drug carrier by culturing the rapamycin-containing drug carrier prepared in step (a) and the stem cell.

According to the present disclosure, it was experimentally confirmed that the stem cell conjugated with the rapamycin-containing drug carrier had an excellent effect of inhibiting the expression of fibrosis-related proteins in pulmonary fibroblasts induced with fibrosis. Therefore, the stem cell conjugated with the rapamycin-containing drug carrier according to the present disclosure may be used in various fields of research related to fibrosis and treatment of pulmonary fibrosis.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1A is a diagram showing results of analyzing the expression of fibrosis-related proteins collagen, α-SMA, and fibronectin according to TGF-β1 treatment in MRC-5 cells induced with fibrosis.

FIG. 1B is a diagram showing results of analyzing the expression of fibrosis-related proteins collagen, α-SMA, and fibronectin according to TGF-β1 treatment in LL97A cells induced with fibrosis.

FIG. 2A is a diagram showing results of confirming cell viability of mesenchymal stem cells UCB #001, UCB #006, and UCB #192 according to rapamycin treatment through CCK-8 assay.

FIG. 2B is a diagram showing results of analyzing HGF secretion of mesenchymal stem cells UCB #001, UCB #006, and UCB #192 according to rapamycin treatment through ELISA.

FIG. 2C is a diagram showing results of analyzing PGE2 secretion of mesenchymal stem cells UCB #001, UCB #006, and UCB #192 according to rapamycin treatment through ELISA.

FIG. 3A is a diagram showing results of confirming cell survival rates of mesenchymal stem cells UCB #001, UCB #006, and UCB #192 according to pirfenidone treatment through CCK-8 assay.

FIG. 3B is a diagram showing results of analyzing HGF secretion of mesenchymal stem cells UCB #001, UCB #006, and UCB #192 according to pirfenidone treatment through ELISA.

FIG. 3C is a diagram showing results of analyzing PGE2 secretion of mesenchymal stem cells UCB #001, UCB #006, and UCB #192 according to pirfenidone treatment through ELISA.

FIG. 4A is a diagram showing results of analyzing the expression of fibrosis-related proteins collagen, α-SMA, and fibronectin according to rapamycin treatment in MRC-5 cells induced with fibrosis.

FIG. 4B is a diagram showing results of quantifying the expression of collagen and α-SMA from the results of FIG. 4A.

FIG. 4C is a diagram showing results of analyzing the expression of fibrosis-related proteins collagen, α-SMA, and fibronectin according to rapamycin treatment in LL97A cells induced with fibrosis.

FIG. 4D is a diagram showing results of quantifying the expression of collagen and α-SMA from the results of FIG. 4C.

FIG. 5A is a diagram showing a contactless co-culturing method for confirming a pulmonary fibrosis inhibitory function of mesenchymal stem cells treated with rapamycin.

FIG. 5B is a diagram showing results of analyzing the expression of fibrosis-related proteins collagen, α-SMA, and fibronectin according to contactless co-culture of MRC-5 cells induced with fibrosis and mesenchymal stem cells treated with rapamycin, rapamycin alone, and mesenchymal stem cells alone.

FIG. 5C is a diagram showing results of quantifying the expression of collagen and α-SMA from the results of FIG. 5B.

FIG. 5D is a diagram showing results of analyzing the expression of fibrosis-related proteins collagen, α-SMA, and fibronectin according to contactless co-culture of LL97A cells induced with fibrosis and mesenchymal stem cells treated with rapamycin, rapamycin alone, and mesenchymal stem cells alone.

FIG. 5E is a diagram showing results of quantifying the expression of collagen and α-SMA from the results of FIG. 5D.

FIG. 6 is a diagram showing results of analyzing the expression of fibrosis-related proteins collagen, α-SMA, and fibronectin according to contactless co-culture of mesenchymal stem cells treated with pirfenidone; and LL97A cells induced with fibrosis.

FIG. 7 is a diagram showing a process of preparing a rapamycin-containing drug carrier by a water-in-oil method.

FIG. 8A is a diagram showing results of observing a rapamycin-containing drug carrier according to the present disclosure using a scanning electron microscope.

FIG. 8B is a diagram showing results of analyzing the size of a rapamycin-containing drug carrier according to the present disclosure using a laser particle analyzer.

FIG. 8C is a diagram showing results of analyzing a drug loading rate of a rapamycin-containing drug carrier according to the present disclosure through HPLC (Theoretical Loading capacity (TLC): Theoretical drug loading rate, Loading capacity (LC): Drug loading rate, Encapsulation efficiency (EE): Percentage of drug loaded in drug carrier compared to total amount of drug used).

FIG. 8D is a diagram showing results of measuring a drug release time of a rapamycin-containing drug carrier according to the present disclosure.

FIG. 9 is a diagram showing results of observing PD-RAP-MS conjugated mesenchymal stem cells according to the present disclosure by microscopy.

FIG. 10A is a diagram showing results of observing mesenchymal stem cells conjugated with a drug-containing drug carrier according to a concentration of a Coumarin-6 fluorescence-labeled drug carrier using confocal microscopy.

FIG. 10B is a diagram showing results of analyzing a drug loading amount on the surface of mesenchymal stem cell according to a concentration of a Coumarin-6 fluorescence-labeled drug carrier using HPLC.

FIG. 10C is a diagram showing results of analyzing a drug release time from the surface of mesenchymal stem cell according to a concentration of a Coumarin-6 fluorescence-labeled drug carrier through LC-MS/MS.

FIG. 10D is a diagram showing results of observing the viability of mesenchymal stem cells conjugated with a drug-containing drug carrier according to a concentration of a drug carrier through AO/PI assay.

FIG. 10E is a diagram showing results of confirming cell viability of mesenchymal stem cells according to a concentration of a drug carrier through CCK-8 assay.

FIG. 10F is a diagram showing results of analyzing the protein expression of Bcl2 and Bax in mesenchymal stem cells according to a concentration of a drug carrier through Western blotting.

FIG. 11A is a diagram showing a method for preparing PD-RAP-MS conjugated mesenchymal stem cells and a contactless co-culturing method thereof according to the present disclosure.

FIG. 11B is a diagram showing results of observing PD-RAP-MS conjugated mesenchymal stem cells according to the present disclosure by microscopy.

FIG. 11C is a diagram showing results of analyzing the expression of fibrosis-related proteins collagen, α-SMA, and fibronectin according to contactless co-culture of MRC-5 cells induced with fibrosis and PD-RAP-MS conjugated mesenchymal stem cells according to the present disclosure, mesenchymal stem cells alone, and rapamycin alone.

FIG. 11D is a diagram showing results of quantifying the expression of collagen and α-SMA from the results of FIG. 11C.

FIG. 11E is a diagram showing results of analyzing the expression of fibrosis-related proteins collagen, α-SMA, and fibronectin according to contactless co-culture of LL97A cells induced with fibrosis and PD-RAP-MS conjugated mesenchymal stem cells according to the present disclosure, mesenchymal stem cells alone, and rapamycin alone.

FIG. 11F is a diagram showing results of quantifying the expression of α-SMA and collagen from the results of FIG. 11E.

### DETAILED DESCRIPTION

Hereinafter, the present disclosure will be described in detail.

According to an aspect of the present disclosure, there is provided a stem cell-drug carrier including a stem cell conjugated with a rapamycin-containing drug carrier on the cell surface.

In the rapamycin-containing drug carrier of the present disclosure, rapamycin is loaded inside the carrier. In the example of the present disclosure, it was confirmed that rapamycin increased the production of anti-fibrotic factors without an adverse affect on the cell viability of stem cells. Unlike this, pirfenidone, which is clinically used as an anti-fibrotic drug, was shown to have an anti-fibrotic function, but found to have no significant effect on changes in secretion of HGF and PGE2 when applied together with the stem cells. In addition, in another example, it was confirmed that rapamycin-treated stem cells significantly inhibited the expression of fibrosis-related proteins, but pirfenidone-treated stem cells did not inhibit the expression of fibrosis-related proteins.

Through this, the present disclosure derived rapamycin as a drug that enhanced the effect of stem cells and enhanced the anti-fibrotic effect together with stem cells when used together with stem cells even among anti-fibrotic drugs. To achieve the anti-fibrotic effect, the stem cells may treated simultaneously with rapamycin, stem cells primed with rapamycin may be used, or stem cells conjugated with a drug carrier containing rapamycin on the cell surface may be used.

The stem cell-drug carrier of the present disclosure is characterized in that the rapamycin-containing drug carrier is conjugated on the cell surface. Due to the technical features above, the stem cell-drug carrier of the present disclosure may not only enable in vivo delivery of stem cells but also improve the release duration of the drug.

In the present disclosure, the drug carrier means a particle formed by forming a polymer coating layer based on the drug, and for convenience, may be indicated in the form of “polymer type-drug carrier”. For example, if the polymer type is poly (lactic-co-glycolic acid) (PLGA), the drug carrier is indicated as a PLGA-drug carrier.

In a specific exemplary embodiment of the present disclosure, the drug carrier may be a biodegradable polymer-drug carrier known in the art. The biodegradable polymer-drug carrier may use biodegradable polymers conjugated with polyethylene glycols of various molecular weights to remain in the body for a long time. Examples of usable biodegradable polymers may be used with polymers selected from the group consisting of polylactide-co-glycolide, polylactide-co-glycolide-co-ethylene glycol, polystyrene-co-ethylene glycol, polyethyleneimine-co-ethylene glycol, polyphosphagen-co-ethylene glycol, polylactide-co-ethylene glycol, polycaprolactone-co-ethylene glycol, polyanhydride-co-ethylene glycol, polymaleic acid-co-ethylene glycol and derivatives thereof, polyalkylcyanoacrylate-co-ethylene glycol, polyhydroxybutyrate-co-ethylene glycol, polycarbonate-co-ethylene glycol and polyorthoester-co-ethylene glycol, polyethylene glycol, poly-L-lysine-co-ethylene glycol, polyglycolide-co-ethylene glycol, polymethylmethacrylate-co-ethylene glycol, polyvinylpyrrolidone-co-ethylene glycol, and copolymers thereof. These polymers are known to exhibit excellent biocompatibility as well as low toxicity.

In a specific exemplary embodiment of the present disclosure, the rapamycin-containing drug carrier may have a size of preferably 0.1 to 10 μm, more preferably 0.5 to 5 μm, and much more preferably 0.7 to 2.9 μm.

In a specific exemplary embodiment of the present disclosure, the rapamycin-containing drug carrier may be coated with polydopamine. The polydopamine coating is used to impart cell adhesion to the rapamycin-containing drug carrier, and in the example of the present disclosure, the polydopamine coating reacted with a Dopamin HCL solution (1.0 mg/mL) under weakly basic conditions (pH 8.0) for 1 hour.

The stem cells of the present disclosure are not limited thereto, but may be autologous or allogenic-derived.

In a specific exemplary embodiment of the present disclosure, the stem cell may be an embryonic stem cell, a mesenchymal stem cell or an induced pluripotent stem cell, and preferably a mesenchymal stem cell.

In the present disclosure, the embryonic stem cell (ESC) is commonly abbreviated as an ES cell, but refer to a cell which is pluripotent and derived from the inner cell mass of a blastocyst, which is an early-stage embryo. For the purpose of the present disclosure, the term “ESC” is also sometimes used broadly and thus includes an embryonic germ cell.

As used in the present invention, the mesenchymal stem cell (MSC) refers to a pluripotent progenitor cell before differentiation into cells of a specific organ, such as bone, cartilage, fat, tendon, nerve tissue, fibroblasts, and muscle cells.

In the present disclosure, the induced pluripotent stem cell (iPSC) is commonly abbreviated as an iPS cell, and refers to a type of normally non-pluripotent cell, such as pluripotent stem cell artificially induced from an adult somatic cell, by inducing the “forced” expression of a specific gene.

In a preferred exemplary embodiment of the present disclosure, the mesenchymal stem cells are preferably derived from embryonic yolk sac, placenta, umbilical cord, umbilical cord blood, skin, peripheral blood, bone marrow, adipose tissue, muscle, liver, nerve tissue, periosteum, fetal membrane, synovium, synovial fluid, amniotic membrane, meniscus, anterior cruciate ligament, articular chondrocytes, milk teeth, perivascular cells, trabecular bone, subpatellar fat pad, spleen or thymus.

The stem cells are preferably derived from humans, but may also be derived from fetuses or mammals other than humans. The mammals other than humans may be more preferably dogs, cats, monkeys, cow, sheep, pig, horse, rat, mouse, guinea pig, or the like, and the origin thereof is not limited.

In the rapamycin-containing drug carrier according to the present disclosure, rapamycin is loaded therein in a spherical shape.

In addition, in the stem cell-drug carrier of the present disclosure, one or more rapamycin-containing drug carriers may be conjugated with the stem cells in single cell units.

In addition, it is preferable that the stem cell-drug carrier of the present disclosure is transplantable into a living body.

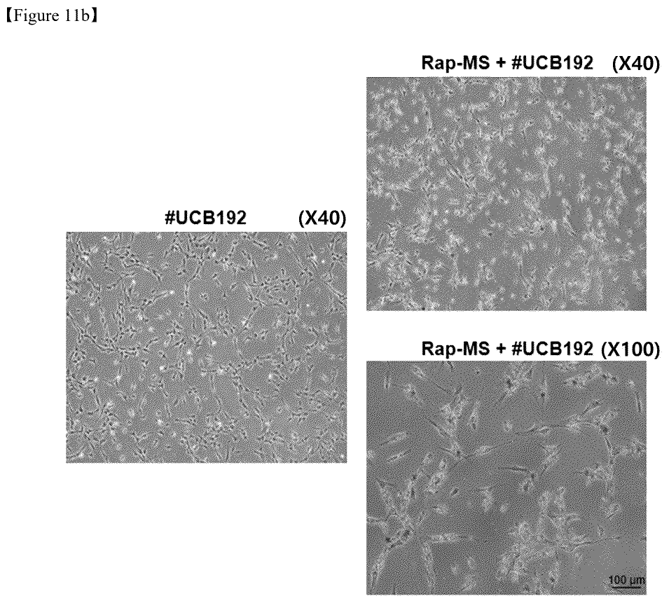

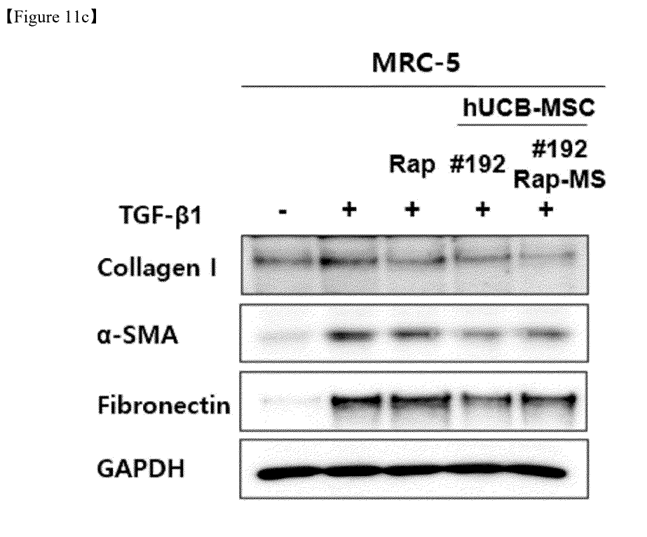

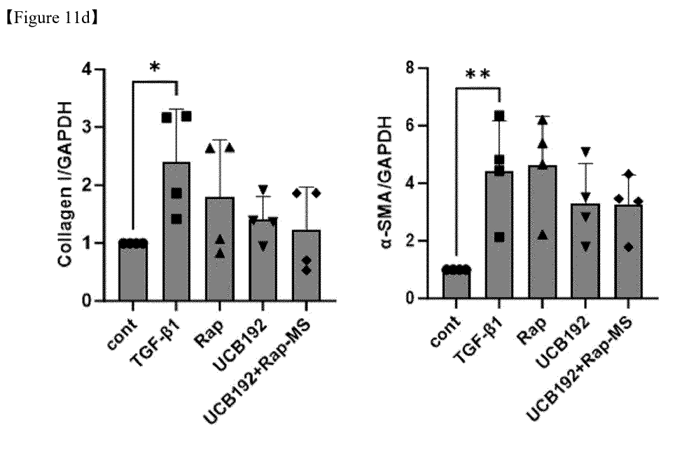

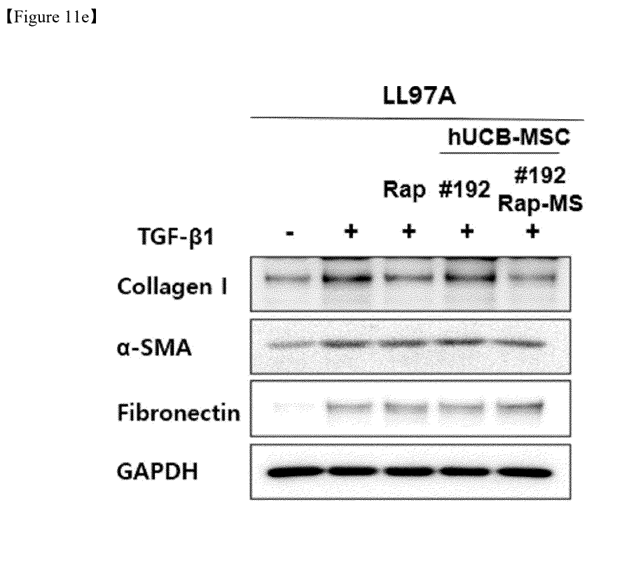

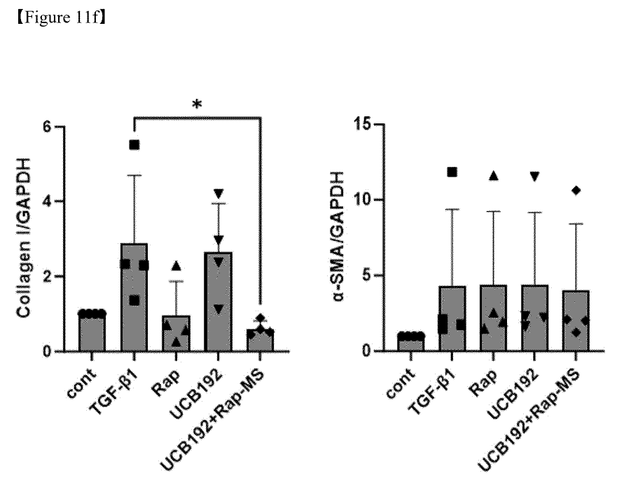

In an example of the present disclosure, the ‘stem cell conjugated with the rapamycin-containing drug carrier on the cell surface’ was fabricated as shown in FIG. 11A. Specifically, a rapamycin-containing drug carrier solution was prepared by dissolving the rapamycin-containing drug carrier in 1.7 ml of PBS. PD-RAP-MS conjugated mesenchymal stem cells were fabricated by culturing the rapamycin-containing drug carrier solution and mesenchymal stem cells for 24 hours.

In a preferred exemplary embodiment of the present disclosure, the concentration of the rapamycin-containing drug carrier solution may be 0.01 to 100 mg/ml, preferably 0.1 to 10 mg/ml, and most preferably 1 mg/ml.

In a preferred exemplary embodiment of the present disclosure, when the rapamycin-containing drug carrier solution and the mesenchymal stem cells are cultured, the mesenchymal stem cells may be contained in an amount of 1×10¹ to 1×10¹⁰ cells/ml, preferably 1×10³ to 1×10⁸ cells/ml, and most preferably 1×10⁶ cells/ml.

According to another aspect of the present disclosure, the present disclosure provides a composition comprising the stem cell-drug carrier.

According to another aspect of the present disclosure, the present disclosure provides a pharmaceutical composition for preventing or treating pulmonary fibrosis. The pharmaceutical composition includes (i) the stem cell-drug carrier; (ii) stem cells primed with rapamycin; or (iii) rapamycin and stem cells.

In an example of the present disclosure, it was confirmed that the stem cells effectively inhibited the expression of fibrosis-related proteins α-SMA, collagen, and fibronectin in pulmonary fibroblasts induced with fibrosis in pulmonary fibrosis. In a subsequent experiment, the present inventors evaluated the effect of rapamycin on the survival rate of stem cells. As a result, it was confirmed that when the stem cells were treated (i.e., primed) with rapamycin, not only the survival rate of the stem cells significantly increased, but also the secretion of HGF and PGE2 was enhanced. The stem cells primed with rapamycin of the present disclosure have improved survival rate, and when co-cultured with fibrosis-induced pulmonary fibroblasts in a contactless manner, the stem cells may significantly contribute to the prevention, improvement, or treatment of pulmonary fibrosis by inhibiting the expression of fibrosis-related proteins.

In a specific exemplary embodiment of the present disclosure, in the stem cells primed with rapamycin, the stem cells may be treated with rapamycin at a concentration of 0.1 to 100 ng/ml, preferably 0.5 to 70 ng/ml, and more preferably 1 to 50 ng/ml.

In a specific exemplary embodiment of the present disclosure, the composition for preventing or treating pulmonary fibrosis including rapamycin and the stem cells may include rapamycin at a concentration of 0.1 to 100 ng/ml, preferably 0.5 to 70 ng/ml, and more preferably 1 to 50 ng/ml.

In the present disclosure, the pulmonary fibrosis refers to a disease in which proliferation of fibrous connective tissue occurs in the lung, resulting in destruction of normal lung structure, and hardening or devastation of lung tissue. The exact cause of pulmonary fibrosis is unknown, but it is known that repeated damage to the alveolar area due to various unknown causes, including viral infection and smoking, induces an inflammatory environment and causes abnormal wound healing responses. That is, when the wound heals, inflammatory cells release profibrotic cytokines such as TGF-β1 that activate fibroblasts, and these factors promote the differentiation of fibroblasts into myofibroblasts, which causes deposition of extracellular matrix components and leads to pulmonary fibrosis.

In a specific exemplary embodiment of the present disclosure, the pulmonary fibrosis may be at least one selected from the group consisting of idiopathic pulmonary fibrosis, pulmonary fibrosis due to anticancer treatment, pulmonary fibrosis due to radiation exposure, pulmonary fibrosis due to infection, pulmonary fibrosis due to drugs, or pneumonia, and may be applied without limitation to diseases caused by pulmonary fibrosis.

The pharmaceutical composition of the present disclosure may be a cell therapy product composition.

As used in the present invention, the cell therapy product refers to a medicine (in US FDA regulations) used for the purposes of treatment, diagnosis, and prevention with cells and tissues prepared through isolation, culture, and special manipulation from a subject, and means a medicine used for treatment, diagnosis and prevention through a series of actions, such as ex vivo proliferating and selecting living autologous, allogeneic, or xenogenic cells to restore the functions of cells or tissues, changing the biological characteristics of cells by other methods, and the like.

The pharmaceutical composition of the present disclosure may be prepared using pharmaceutically suitable and physiologically acceptable adjuvants in addition to the active ingredients. As the adjuvants, excipients, disintegrants, sweeteners, binders, coating agents, expanding agents, lubricants, slip modifiers, or flavoring agents may be used.

The composition of the present disclosure may be formulated as a pharmaceutical composition by further including at least one pharmaceutically acceptable carrier in addition to the above-described active ingredients for administration. The pharmaceutically acceptable carrier may be used by mixing saline, sterile water, Ringer's solution, buffered saline, a dextrose solution, a maltodextrin solution, glycerol, ethanol, liposomes, and at least one of these ingredients, and if necessary, other conventional additives such as an antioxidant, a buffer, a bacteriostat, etc. may be added. In addition, the composition may be formulated in injections such as aqueous solutions, suspensions and emulsions, pills, capsules, granules or tablets by further adding diluents, dispersants, surfactants, binders and lubricants, and target organ-specific antibodies or other ligands may be used in combination with the carrier so as to specifically act to the target organ. Furthermore, the composition may be preferably formulated according to each disease or ingredients using an appropriate method in the art or methods disclosed in Remington's literature.

The pharmaceutical composition of the present disclosure may be in the form of a solution, a suspension, a dispersion, an emulsion, a gel agent, an injectable solution, and a sustained-release preparation of an active compound, and preferably an injection.

When the pharmaceutical composition of the present disclosure is formulated as the injection, the pharmaceutical composition may be formulated in a physically and chemically very stable injection by adjusting pH using a buffer solution such as an acid aqueous solution or a phosphate solution that may be used as an injection to ensure product stability according to distribution of the injection prescription.

More specifically, the injection may be prepared by dissolving the pharmaceutical composition in water for injection together with a stabilizer or a solubilizing agent, and then sterilizing the pharmaceutical composition, particularly, by high-temperature and reduced-pressure sterilization or aseptic filtration. The water for injection may be distilled water for injection or a buffer solution for injection, for example, a phosphate buffer solution or a sodium dihydrogen phosphate (NaH₂PO₄)-citric acid buffer solution in a pH range of 3.5 to 7.5. The phosphate used may be in the form of a sodium or potassium salt, or an anhydrous or hydrated product, and may also be in the form of citric acid, or an anhydrous or hydrated product.

In addition, the stabilizer used in the present disclosure includes sodium pyrosulfite, sodium bisulfite, sodium metabisulfite or ethylenediamine tetra acetic acid, and the solubilizing agent includes base such as sodium hydroxide, sodium bicarbonate, sodium carbonate or potassium hydroxide, or acid such as hydrochloric acid or acetic acid.

The injection according to the present disclosure may be formulated to be bioabsorbable, biodegradable, and biocompatible. Bioabsorbability means that the injection may disappear from the body upon initial application, with or without degradation of the dispersed injection. Biodegradability means that the injection may be broken down or degraded in the body by hydrolysis or enzymatic degradation. Biocompatibility means that all of the ingredients are non-toxic in the body.

The injection according to the present disclosure may be prepared using a conventional diluent such as a filler, an extender, a binder, a wetting agent, and a surfactant, or an excipient.

The composition or effective ingredient of the present disclosure may be administered by a conventional method, depending on the purpose, via intravenous, intraarterial, intraperitoneal, intramuscular, intrasternal, transdermal, intranasal, subcutaneous, intrauterine epidural, inhalation, topical, rectal, oral, intraocular, or intradermal route, and preferably administered intravenously. The composition or active ingredient of the present disclosure may be administered by an injection or catheter.

In the composition of the present disclosure, the dose of the active ingredient may be adjusted within a range of 1×10¹ to 1×10⁵⁰ units/kg, preferably 1×10¹ to 1×10³⁰ units/kg, more preferably 1×10⁵ to 1×10²⁰ units/kg, and most preferably 1×10⁷ to 1×10⁹ units/kg for an adult weighing 60 kg. However, an optimal dose to be administered may be easily determined by those skilled in the art, and may be adjusted according to various factors including a type of disease, the severity of disease, the contents of active ingredients and other ingredients contained in the composition, a type of formulation, age, body weight, general health condition, sex and diet of a patient, an administration time, a route of administration, a secretion rate of a composition, duration of treatment, and drugs to be used concurrently.

According to yet another aspect of the present disclosure, the present disclosure provides a reagent composition for inhibiting fibrosis. The reagent composition includes (i) the stem cell-drug carrier; (ii) stem cells primed with rapamycin; or (iii) rapamycin and stem cells.

According to yet another aspect of the present disclosure, the present disclosure provides a method for treatment or inhibiting of fibrosis, comprising administering (i) the stem cell-drug carrier; (ii) stem cells primed with rapamycin; or (iii) rapamycin and stem cells; to a subject in need thereof.

In a specific exemplary embodiment of the present disclosure, it was confirmed that the stem cell-drug carrier inhibited the protein expression of fibrosis-related protein Alpha-smooth muscle actin (α-SMA) or collagen in fibrosis-induced pulmonary fibroblasts. In addition, it was experimentally confirmed that the stem cell-drug carrier significantly improved the duration of drug release. Therefore, the stem cell-drug carrier of the present disclosure may be used in various fields of research related to fibrosis and treatment of pulmonary fibrosis.

The reagent composition of the present disclosure may further include one or more known ingredients having a fibrosis inhibitory effect.

In a specific exemplary embodiment of the present disclosure, the fibrosis may be at least one selected from the group consisting of idiopathic pulmonary fibrosis, pulmonary fibrosis due to anticancer treatment, pulmonary fibrosis due to radiation exposure, pulmonary fibrosis due to infection, pulmonary fibrosis due to drugs, or pneumonia, and may be applied without limitation to diseases caused by pulmonary fibrosis.

According to yet another aspect of the present disclosure, there is provided a method for preparing a cell therapy product for preventing or treating pulmonary fibrosis including (a) preparing a rapamycin-containing drug carrier by mixing and homogenizing rapamycin and a polymer; and (b) preparing a stem cell conjugated with the rapamycin-containing drug carrier by culturing the rapamycin-containing drug carrier prepared in step (a) and the stem cell.

In a specific exemplary embodiment of the present disclosure, the polymer of step (a) may be a biodegradable polymer as described above.

In a specific exemplary embodiment of the present disclosure, step (a) may be mixing and homogenizing an aqueous solution; and an oily solution containing rapamycin and a polymer.

In a specific exemplary embodiment of the present disclosure, step (a) may further include coating the prepared rapamycin-containing drug carrier with polydopamine.

Duplicated contents are omitted in consideration of the complexity of the present specification, and terms not defined otherwise in the present specification have the meanings commonly used in the art to which the present disclosure pertains.

Hereinafter, the present disclosure will be described in more detail through Examples. These Examples are just illustrative of the present disclosure, and it will be apparent to those skilled in the art that it is not interpreted that the scope of the present disclosure is limited to these Examples.

### Example 1. Confirmation of Pulmonary Fibrosis Inhibitory Function of Mesenchymal Stem Cells

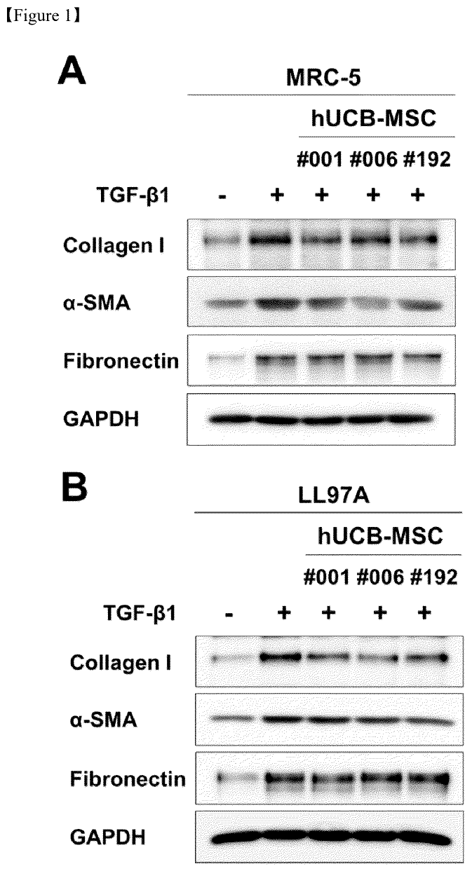

The pulmonary fibrosis inhibitory effect of mesenchymal stem cells was evaluated. Specifically, normal pulmonary fibroblasts (MRC-5) and idiopathic pulmonary fibrosis patient-derived pulmonary fibroblasts (LL97A) were treated with TGF-β1 (10 ng/ml) for 24 hours to induce myofibroblast differentiation (i.e., fibrosis induction). First, fibrosis-induced cells and mesenchymal stem cells UCB #001, UCB #006, and UCB #192 were co-cultured in a contactless manner for 24 hours. The expression of fibrosis-related proteins collagen, α-SMA, and fibronectin, known to increase upon TGF-β1 treatment after co-culture, was analyzed by Western blotting. The results of analyzing the expression of fibrosis-related proteins in the co-cultured MRC-5 and LL97A cells were shown in FIGS. 1A and 1B, respectively.

As shown in FIGS. 1A and 1B, the expression of collagen, α-SMA, and fibronectin in the cultured MRC-5 and LL97A cells was decreased according to co-culture, and in particular, the expression of collagen was significantly reduced.

### Example 2. Confirmation of Increased Survival Rate of Mesenchymal Stem Cells and Secretion of HGF and PGE2 by Treatment With Rapamycin

### 2-1. Rapamycin

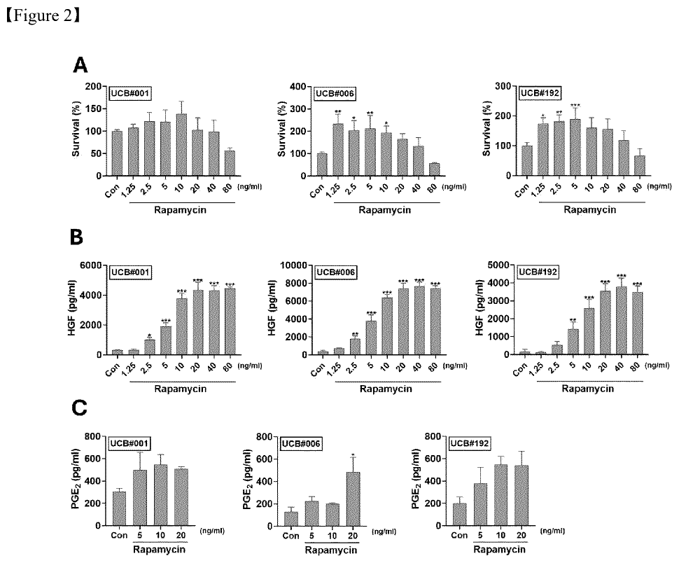

The effects of rapamycin on the survival rate of mesenchymal stem cells and the secretion of HGF and PGE2 were confirmed. Specifically, the mesenchymal stem cells UCB #001, UCB #006, and UCB #192 were treated with rapamycin at various concentrations of 1.25, 2.5, 5, 10, 20, 40, and 80 ng/ml for 24 hours. The cell viability of cells treated with rapamycin was confirmed by CCK-8 assay, and the results were shown in FIG. 2A.

As shown in FIG. 2A, it was confirmed that when the mesenchymal stem cells were treated with rapamycin at a concentration of 1.25 to 40 ng/ml, the cell viability was increased and cytotoxicity was exhibited at concentrations of 80 ng/ml or higher.

In addition, changes in HGF and PGE2 secretion in mesenchymal stem cells treated with rapamycin at various concentrations were analyzed by ELISA. The results of analyzing HGF and PGE2 secretion were shown in FIGS. 2B and 2C, respectively.

As shown in FIGS. 2B and 2C, it was confirmed that in the mesenchymal stem cells treated with rapamycin, the secretion of HGF increased in a rapamycin concentration-dependent manner, and the secretion of PGE2 also significantly increased according to rapamycin treatment.

The results mean that rapamycin at a concentration of about 20 ng/ml may increase the production of anti-fibrotic factors without an adverse effect on the cell viability of mesenchymal stem cells.

### 2-2. Pirfenidone

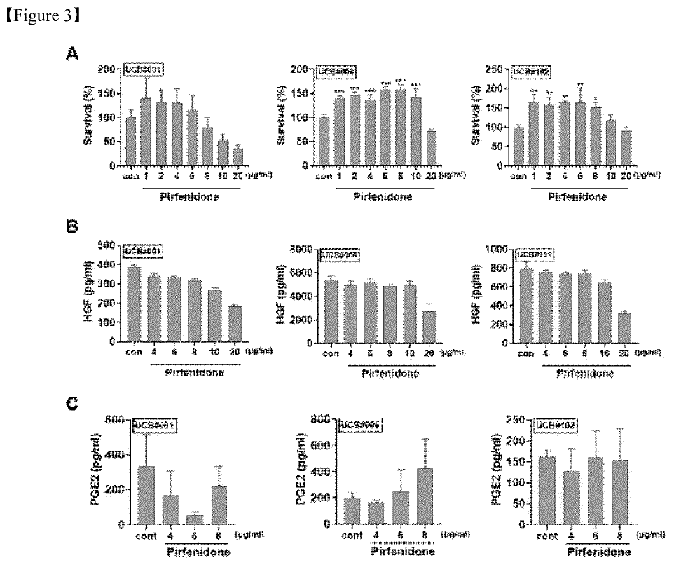

The effects of pirfenidone, a drug used clinically as an anti-fibrotic drug, on the survival rate of mesenchymal stem cells and the secretion of HGF and PGE2 were confirmed in the same manner as rapamycin. The results of confirming the survival rate of mesenchymal stem cells according to pirfenidone treatment were shown in FIG. 3A, and the results of analyzing HGF and PGE2 secretion were shown in FIGS. 3B and 3C, respectively.

As shown in FIG. 3A, it was confirmed that in UCB #001, the cell viability increased when treated with pirfenidone at a concentration of 1 to 6 μg/ml. In addition, it was confirmed that in UCB #006 and UCB #192, the cell viability increased when treated at a concentration of 1 to 10 μg/ml, and cytotoxicity was exhibited at 20 μg/ml or higher.

As shown in FIGS. 3B and 3C, it was confirmed that pirfenidone treated at 4 to 10 g/ml did not affect HGF secretion, but pirfenidone treated at 20 μg/ml reduced HGF secretion. In addition, there was no significant change in PGE2 secretion according to pirfenidone treatment.

The results mean that as confirmed in Example 2-1, rapamycin treatment induces a significant increase in the secretion of HGF and PGE2, whereas pirfenidone does not have the same function as rapamycin. This suggests that even drugs with an anti-fibrotic function may have different effects on mesenchymal stem cells, which may lead to a difference in effect when primed or co-administered.

### Example 3. Confirmation of Pulmonary Fibrosis Inhibitory Function of Rapamycin

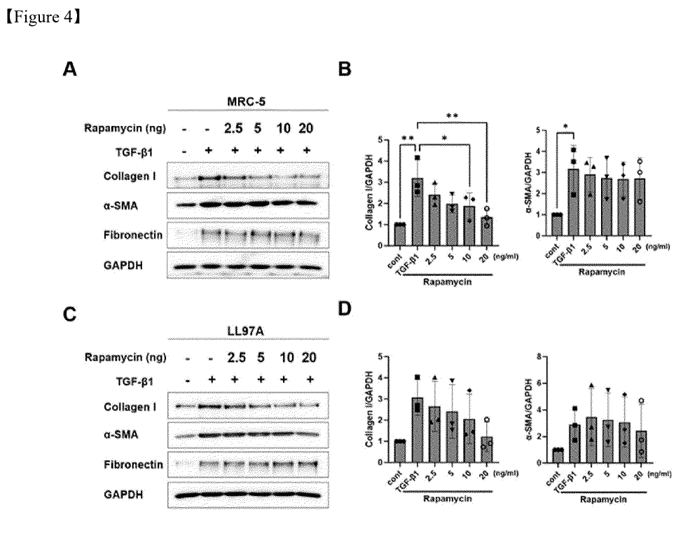

In Example 2, it was confirmed that rapamycin increased the anti-fibrosis-related function of mesenchymal stem cells. In Example, it was confirmed whether treatment of rapamycin alone exhibited an anti-fibrotic effect. Specifically, normal pulmonary fibroblasts (MRC-5) and idiopathic pulmonary fibrosis patient-derived pulmonary fibroblasts (LL97A) were treated with TGF-β1 for 24 hours to induce fibrosis. The fibrosis-induced cells were treated with rapamycin at various concentrations of 2.5, 5, 10, and 20 ng/ml for 24 hours. Thereafter, the expression of fibrosis-related proteins collagen, α-SMA, and fibronectin was analyzed through Western blotting. In addition, the expression of collagen and α-SMA was quantified using the Image J program. The results of analyzing the expression of fibrosis-related proteins in MRC-5 cells were shown in FIGS. 4A and 4B, and the results of analyzing the expression of fibrosis-related proteins in LL97A cells were shown in FIGS. 4C and 4D.

As shown in FIGS. 4A to 4D, it was confirmed that collagen expression, which had been increased by anti-fibrotic induction in a concentration-dependent manner, was decreased in both the cells. In both the cells, it was confirmed that the expression of α-SMA did not change significantly, but decreased slightly when treated at a concentration of 20 ng/mL.

Example 4. Confirmation of Pulmonary Fibrosis Inhibitory Function of Mesenchymal Stem Cells Treated With Rapamycin

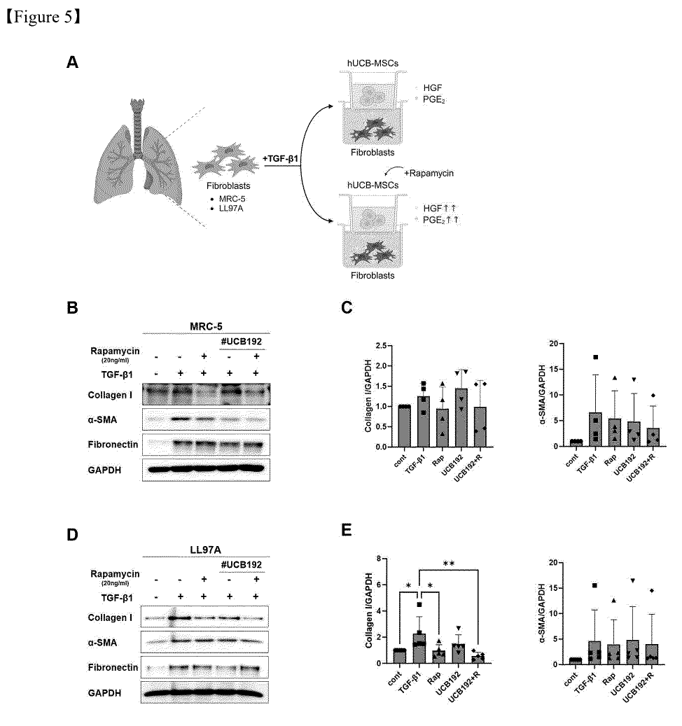

In previous Examples, it was confirmed that rapamycin increased the anti-fibrotic related function of mesenchymal stem cells and exhibited an anti-fibrotic effect alone. In Example, the pulmonary fibrosis inhibitory function of mesenchymal stem cells treated with rapamycin was confirmed as shown in FIG. 5A. Specifically, normal pulmonary fibroblasts (MRC-5) and idiopathic pulmonary fibrosis patient-derived pulmonary fibroblasts (LL97A) were treated with TGF-β1 for 24 hours to induce fibrosis.

Mesenchymal stem cells (UCB #192) and rapamycin were co-cultured with fibrosis-inducing cells in a contactless manner for 24 hours (Experimental Group, UCB192+R). In addition, a control group was treated with rapamycin alone (Rap) or mesenchymal stem cells alone (UCB192). Thereafter, the expression of fibrosis-related proteins α-SMA, collagen, and fibronectin in each group was analyzed through Western blotting, and the expression of collagen and α-SMA was quantified using the Image J program. The results of analyzing the expression of fibrosis-related proteins in MRC-5 cells were shown in FIGS. 5B and 5C, and the results of analyzing the expression of fibrosis-related proteins in LL92A cells were shown in FIGS. 5D and 5E.

As shown in FIGS. 5B to 5E, MRC-5 cells and LL97A cells showed increased protein expression of α-SMA and collagen according to TGF-β1 treatment. However, when rapamycin and the mesenchymal stem cells were treated together (UCB192+R), it was confirmed that the protein expression of collagen and α-SMA was reduced compared to the control group (Rap group, UCB192 group).

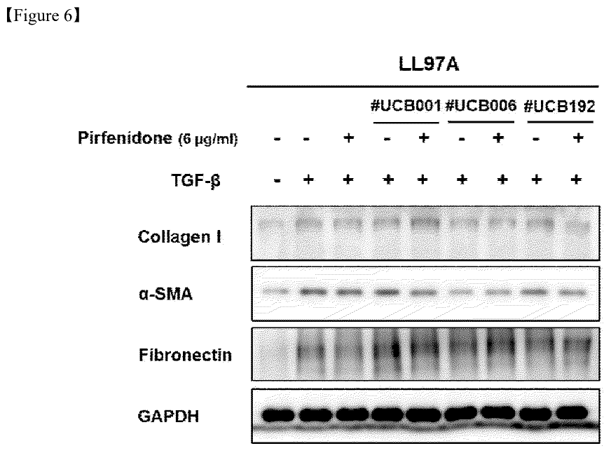

Meanwhile, it was further examined whether combined treatment with the anti-fibrotic drug pirfenidone and mesenchymal stem cells UCB001, UCB006, and UCB192 in LL97A cells affected the expression of fibrosis-related proteins α-SMA, collagen, and fibronectin in LL97A cells. This was performed in the same manner as the rapamycin described above. The results of analyzing the expression of fibrosis-related proteins according to combined treatment with pirfenidone and mesenchymal stem cells were shown in FIG. 6.

As shown in FIG. 6, it was confirmed that the protein expression of collagen and α-SMA increased according to TGF-β1 treatment in LL97A cells. It was confirmed that the combined treatment group of the pirfenidone and mesenchymal stem cells UCB001, UCB006, and UCB 192 showed almost no change in the expression of collagen and α-SMA despite the combined treatment with mesenchymal stem cells.

The results indicate that although pirfenidone is the anti-fibrotic drug, there is no significant synergistic effect when used in combination with mesenchymal stem cells.

### Example 5. Fabrication of Rapamycin-Containing Drug Carrier

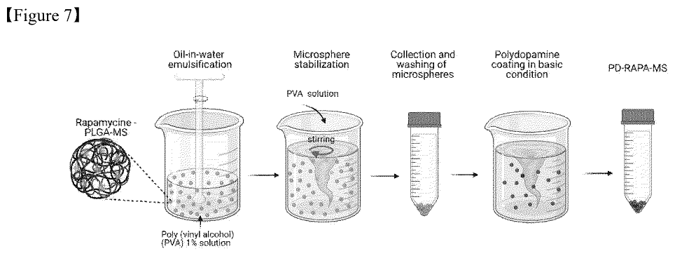

A rapamycin-containing drug carrier was prepared using a water-in-oil method using poly (lactic-co-glycolic acid) (PLGA) (FIG. 7). Specifically, 1 mL of dichloromethane (DCM) (oil phase) dissolved with PLGA and rapamycin was added to 5 mL of a 1% poly (vinyl alcohol) (PVA) solution (water phase) and homogenized at 21,000 rpm for 4 minutes. The homogenized solution was stabilized in 80 mL of the water phase for 4 hours and then washed. The washed drug carrier reacted with a Dopamine HCL solution (1.0 mg/mL) under weakly basic conditions (pH 8.0) for 1 hour to impart cell adhesion. After the reaction, unbound polydopamine was washed several times and removed to obtain a rapamycin-containing drug carrier coated with polydopamine (PD-RAP-MS). The obtained rapamycin-containing drug carrier was then lyophilized and stored.

### Example 6. Characteristic Analysis of Rapamycin-Containing Drug Carrier

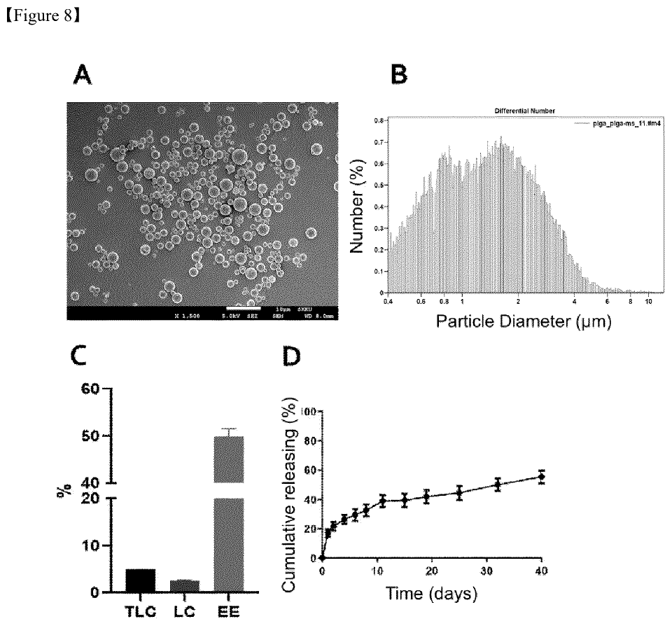

The rapamycin-containing drug carrier prepared in Example 5 above was observed using a scanning electron microscope (SEM), and the results were shown in FIG. 8A.

As shown in FIG. 8A, it was confirmed that the rapamycin-containing drug carrier had a rough spherical shape.

The size of the rapamycin-containing drug carrier was analyzed using a laser particle analyzer, and the results were shown in FIG. 8B.

As shown in FIG. 8B, it was confirmed that the rapamycin-containing drug carrier had an average size of 1.76+1.05 μm.

The loading capacity of a drug (i.e., rapamycin) of the rapamycin-containing drug carrier was analyzed by HPLC, and the results were shown in FIG. 8C.

As shown in FIG. 8C, it was confirmed that the rapamycin-containing drug carrier had approximately 2.49% of the drug (rapamycin) loaded in the carrier.

Finally, the drug release of the rapamycin-containing drug carrier was confirmed in a drug release solution (PBS, 1% Tween 20). The results of analyzing the drug release of the rapamycin-containing drug carrier were shown in FIG. 8D.

As shown in FIG. 8D, it was confirmed that the rapamycin-containing drug carrier released 55% of the drug for 40 days.

### Example 7. Fabrication of Rapamycin Drug Carrier-Conjugated Mesenchymal Stem Cells

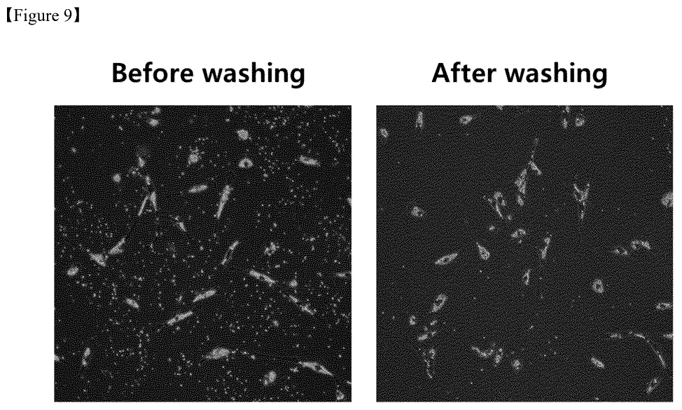

In Example, stem cells conjugated with the rapamycin-containing drug carrier (PD-RAP-MS) prepared in Example 5 on the cell surface were fabricated. Specifically, the mesenchymal stem cells were washed three times with PBS, and then 1.0×10⁶ mesenchymal stem cells were resuspended in 1.7 ml of HBSS (pH 8.0). The resuspended mesenchymal stem cells were stirred for 10 minutes at various concentrations (0.1, 0.2, 0.5, and 1.0 mg/mL) of the rapamycin-containing drug carrier and then cultured in a cell culture dish for 24 hours. To remove the rapamycin-containing drug carrier that was not conjugated to the mesenchymal stem cells after culture, the cell culture dish was washed with PBS. Thereafter, ‘PD-RAP-MS conjugated mesenchymal stem cells’ were finally obtained. The obtained PD-RAP-MS conjugated mesenchymal stem cells were observed visually under a microscope, and the results were shown in FIG. 9.

As shown in FIG. 9, it was confirmed that the rapamycin-containing drug carrier that was not conjugated to the mesenchymal stem cells was removed by washing with PBS. The cells present in the cell culture dish after washing were ‘PD-RAP-MS conjugated mesenchymal stem cells.’

### Example 8. Optimization of PD-RAP-MS Conjugated Mesenchymal Stem Cells

To optimize the conjugation of a rapamycin-containing drug carrier and mesenchymal stem cells, an experiment was performed using Coumarin-6, which was used as a fluorescent probe in a microparticle drug delivery system. Specifically, the PD-RAP-MS fabricated in Example 5 was labeled with Coumarin-6 to prepare a ‘Coumarin-6 fluorescence-labeled drug carrier’. The prepared Coumarin-6 fluorescence-labeled drug carrier was mixed and stirred with mesenchymal stem cells at various concentrations of 0.1, 0.2, 0.5, and 1.0 mg/ml to fabricate ‘Coumarin-6 labeled drug carrier-conjugated mesenchymal stem cells’. The characteristics of the prepared Coumarin-6 labeled drug carrier-conjugated mesenchymal stem cells were analyzed.

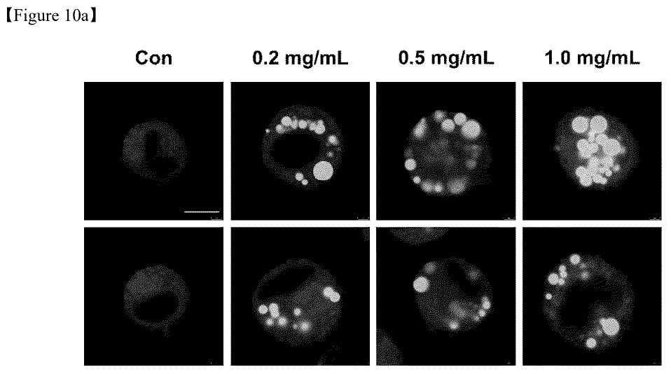

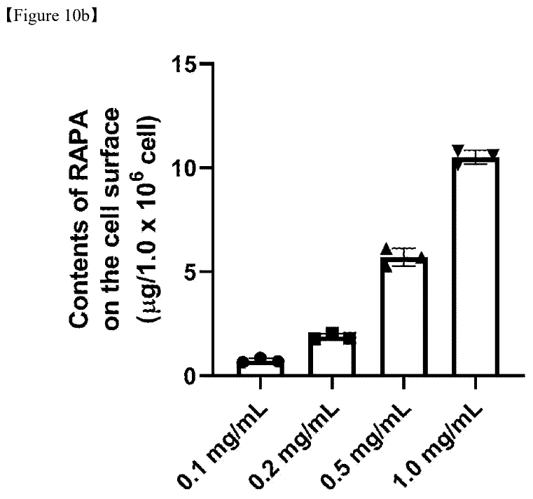

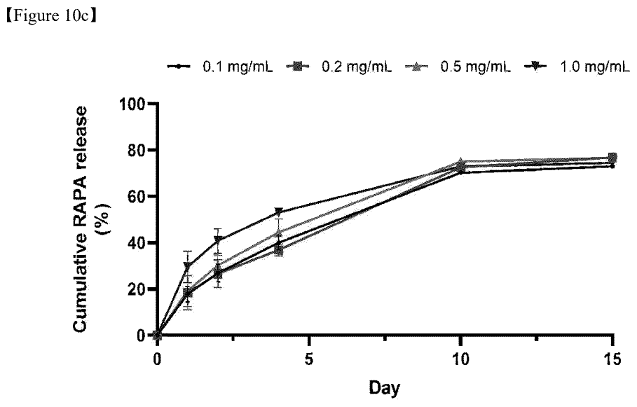

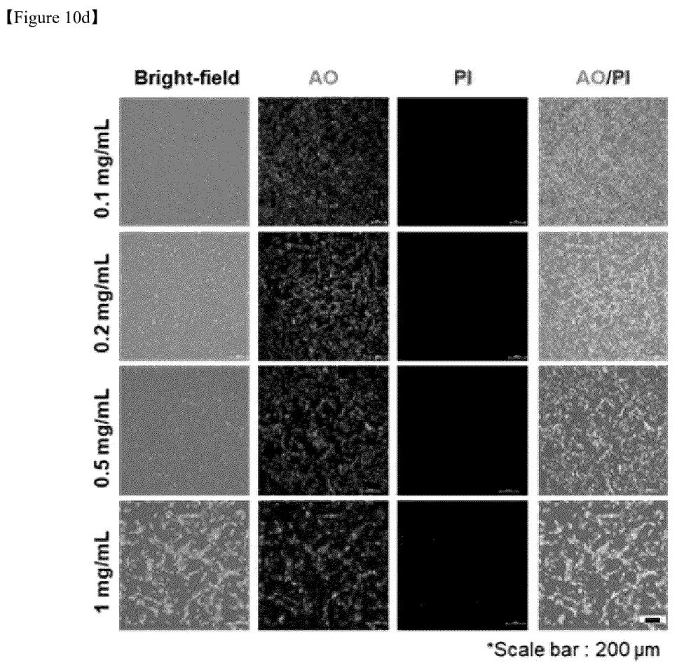

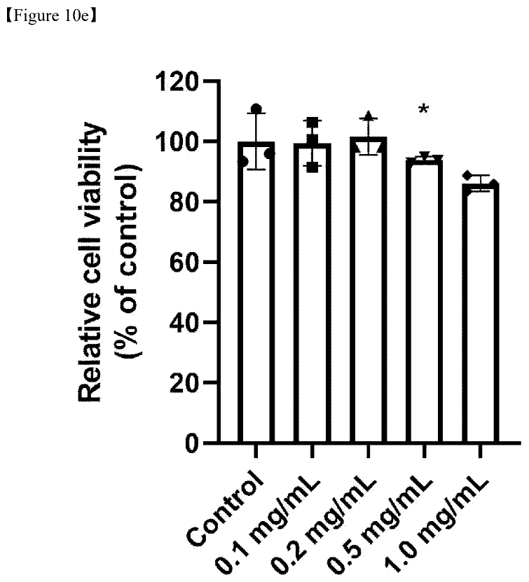

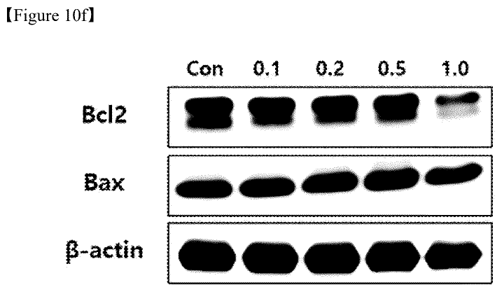

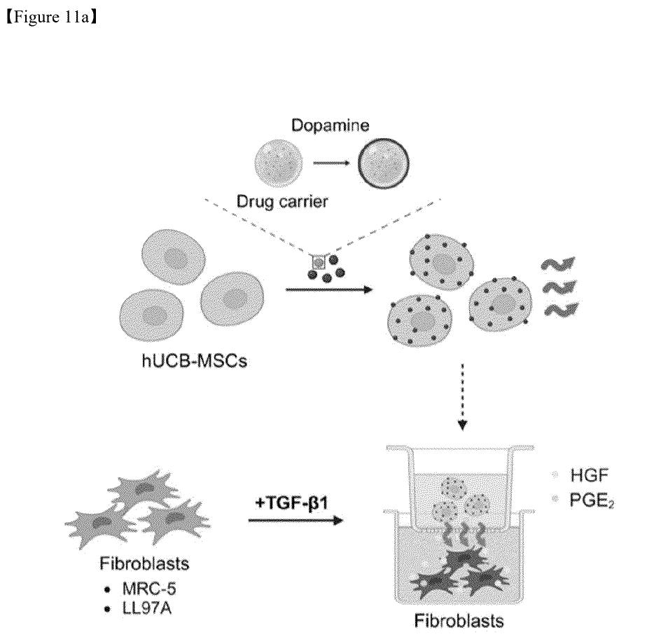

The Coumarin-6 fluorescence-labeled drug carrier-conjugated mesenchymal stem cells were observed using confocal microscopy (CLSM). Stem cells of mesenchymal stem cells were observed in red, and the Coumarin-6 fluorescence-labeled drug carrier was observed in green. The results of observation using a confocal microscope were shown in FIG. 10A.

As shown in FIG. 10A, it was confirmed that the Coumarin-6 fluorescence-labeled drug carrier was uniformly conjugated to the surface of mesenchymal stem cells.

In addition, the rapamycin loading amount of Coumarin-6 fluorescence-labeled drug carrier-conjugated mesenchymal stem cells was confirmed. Specifically, the Coumarin-6 fluorescence-labeled drug carrier-conjugated mesenchymal stem cells were dissolved in acetonitrile and then the supernatant was separated. Rapamycin included in the separated supernatant was detected using HPLC. The results of analyzing the drug loading amount (i.e., the amount of drug on the cell surface) according to the concentration of the Coumarin-6 fluorescence-labeled drug carrier were shown in FIG. 10B.

As shown in FIG. 10B, it was confirmed that as the concentration of the Coumarin-6 fluorescence-labeled drug carrier increased, the amount of rapamycin on the cell surface also increased.

The amount of drug released over time from the Coumarin-6 fluorescence-labeled drug carrier-conjugated mesenchymal stem cells was analyzed using LC-MS/MS. The drug release amount over time was shown in FIG. 10C.

As shown in FIG. 10C, it was confirmed that the Coumarin-6 fluorescence-labeled drug carrier-conjugated mesenchymal stem cells released rapamycin for about 15 days or more.

The survival rate and proliferation capacity of the drug carrier-conjugated mesenchymal stem cells were further analyzed using AO/PI assay, CCK-8 assay, and Western blot. The results of AO/PI assay, CCK-8 assay, and Western blot were shown in FIGS. 10D to 10F, respectively.

As shown in FIGS. 10D to 10F, as the concentration of the drug carrier increased, the survival rate and proliferation capacity of stem cells decreased, but the degree of decrease was not significant.

### Example 9. Confirmation of Pulmonary Fibrosis Inhibitory Function of PD-RAP-MS Conjugated Mesenchymal Stem Cells

As shown in FIG. 11A, a rapamycin-containing drug carrier solution was prepared by dissolving 1.7 mg of a rapamycin-containing drug carrier in 1.7 ml of PBS. The concentration of the rapamycin-containing drug carrier solution was 1 mg/ml. PD-RAP-MS conjugated mesenchymal stem cells were fabricated by culturing the rapamycin-containing drug carrier solution and 1×10⁶ cells/ml of mesenchymal stem cells (#UCB192) for 24 hours.

The fabricated PD-RAP-MS conjugated mesenchymal stem cells were observed under a microscope, and the results were shown in FIG. 11B.

As shown in FIG. 11B, it was confirmed that in PD-RAP-MS conjugated mesenchymal stem cells (Rap-MS+#UCB192), the rapamycin-containing drug carrier and the mesenchymal stem cells were conjugated well.

Pulmonary fibroblasts MRC-5 and LL97A were treated with TGF-β1 for 24 hours to induce fibrosis. The fibrosis-induced pulmonary fibroblasts; and the PD-RAP-MS conjugated mesenchymal stem cells were co-cultured in a contactless manner (FIG. 11A). After contactless co-culture, the expression of fibrosis-related proteins collagen, α-SMA, and fibronectin was confirmed through Western blotting. The results of Western blotting using MRC-5 cells were shown in FIGS. 11C and 11D. In addition, the Western blotting results using LL97A cells were shown in FIGS. 11E and 11F.

As shown in FIGS. 11C to 11F, it was confirmed that when the two cells and the PD-RAP-MS conjugated mesenchymal stem cells were co-cultured in a contactless manner, the expression of α-SMA and collagen increased by TGF-β1 was significantly reduced.

In summary, the present inventors confirmed that rapamycin served as a priming agent to increase the production of anti-fibrotic factors without affecting the cell viability of mesenchymal stem cells. Thereafter, a rapamycin-containing drug carrier coated with polydopamine was prepared and mixed with mesenchymal stem cells to finally obtain ‘PD-RAP-MS conjugated mesenchymal stem cells’. The PD-RAP-MS conjugated mesenchymal stem cells had a rapamycin-containing drug carrier conjugated to the surface of the mesenchymal stem cells. It was experimentally confirmed that the PD-RAP-MS conjugated mesenchymal stem cells had an excellent effect in inhibiting the expression of fibrosis-related proteins in fibrosis-induced pulmonary fibroblasts and the drug release duration was significantly longer. Therefore, PD-RAP-MS conjugated mesenchymal stem cells may be used in various fields of research related to fibrosis and treatment of pulmonary fibrosis.

As described above, specific parts of the present disclosure have been described in detail, and it will be apparent to those skilled in the art that these specific techniques are merely preferred exemplary embodiments, and the scope of the present disclosure is not limited thereto. Therefore, the substantial scope of the present disclosure will be defined by the appended claims and their equivalents.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A stem cell-drug carrier comprising a stem cell conjugated with a rapamycin-containing drug carrier on the cell surface.

2. The stem cell-drug carrier of claim 1, wherein the rapamycin-containing drug carrier is prepared with one or more polymers selected from the group consisting of polylactide-co-glycolide, polylactide-co-glycolide-co-ethylene glycol, polystyrene-co-ethylene glycol, polyethyleneimine-co-ethylene glycol, polyphosphagen-co-ethylene glycol, polylactide-co-ethylene glycol, polycaprolactone-co-ethylene glycol, polyanhydride-co-ethylene glycol, polymaleic acid-co-ethylene glycol and derivatives thereof, polyalkylcyanoacrylate-co-ethylene glycol, polyhydroxybutyrate-co-ethylene glycol, polycarbonate-co-ethylene glycol and polyorthoester-co-ethylene glycol, polyethylene glycol, poly-L-lysine-co-ethylene glycol, polyglycolide-co-ethylene glycol, polymethylmethacrylate-co-ethylene glycol, polyvinylpyrrolidone-co-ethylene glycol, and copolymers thereof.

3. The stem cell-drug carrier of claim 1, wherein the size of the rapamycin-containing drug carrier is 0.1 to 10 μm.

4. The stem cell-drug carrier of claim 1, wherein the rapamycin-containing drug carrier is coated with polydopamine.

5. The stem cell-drug carrier of claim 1, wherein the stem cell is an embryonic stem cell, a mesenchymal stem cell or an induced pluripotent stem cell.

6. The stem cell-drug carrier of claim 5, wherein the mesenchymal stem cell is derived from embryonic yolk sac, placenta, umbilical cord, umbilical cord blood, skin, peripheral blood, bone marrow, adipose tissue, muscle, liver, nerve tissue, periosteum, fetal membrane, synovium, synovial fluid, amniotic membrane, meniscus, anterior cruciate ligament, articular chondrocytes, milk teeth, perivascular cells, trabecular bone, subpatellar fat pad, spleen or thymus.

7. A composition comprising the stem cell-drug carrier according to claim 1.

8. The composition of claim 7, wherein the composition is a pharmaceutical or reagent composition.

9. The composition of claim 7, wherein the composition is for treating pulmonary fibrosis or inhibiting fibrosis.

10. The composition of claim 9, wherein the pulmonary fibrosis is at least one selected from the group consisting of idiopathic pulmonary fibrosis, pulmonary fibrosis due to anticancer treatment, pulmonary fibrosis due to radiation exposure, pulmonary fibrosis due to infection, pulmonary fibrosis due to drugs, or pneumonia.

11. The composition of claim 9, wherein the stem cell-drug carrier inhibits the protein expression of fibrosis-related protein collagen or Alpha-smooth muscle actin (α-SMA).

12. A method for treatment or inhibiting of fibrosis, comprising administering the stem cell-drug carrier according to claim 1 to a subject in need thereof.

13. A method for treatment or inhibiting of fibrosis, comprising administering a stem cell primed with rapamycin to a subject in need thereof.

14. A method for treatment or inhibiting of fibrosis, comprising administering a rapamycin and a stem cell to a subject in need thereof.

15. A method for preparing a cell therapy product for preventing or treating pulmonary fibrosis comprising:
(a) preparing a rapamycin-containing drug carrier by mixing and homogenizing rapamycin and a polymer; and
(b) preparing a stem cell conjugated with the rapamycin-containing drug carrier by culturing the rapamycin-containing drug carrier prepared in step (a) and the stem cell.
